(19) United States
(12) Patent Application Publication
(10) Pub. No.: US 2020/0309771 A1
(43) Pub. Date: Oct. 1, 2020

(54) BIOSENSOR MANUFACTURE

(71) Applicant: ELISHA SYSTEMS LIMITED, Macclesfield Cheshire (GB)

(72) Inventors: Timothy David Gibson, Wakefield Yorkshire (GB); Duncan William Mackenzie Sharp, Althorpe North Lincolnshire (GB)

(21) Appl. No.: 16/760,980

(22) PCT Filed: Oct. 30, 2018
(86) PCT No.: PCT/EP2018/079742

§ 371 Date: May 1, 2020

(30) Foreign Application Priority Data
Nov. 2, 2017 (EP) 17199766.1

(51) Int. Cl. G01N 33/543; C09D 11/037; C09D 11/322; C09D 11/52; C12Q 1/00

(52) U.S. Cl. CPC G01N 33/5438; C09D 11/037; C12Q 1/001; C09D 11/52; C09D 11/322

(57) ABSTRACT

The manufacture of an electrochemical sensor precursor comprising the steps of: preparing an electrically conductive biocomposite comprising an aqueous mixture of:
polycationic conductive polymer particles;
an anionic polyelectrolyte;
or pre-existing composites of polycationic conducting polymers and anionic polyelectrolytes
a biological affinity agent; and
an optional buffer;
applying the mixture to a region of an electroconductive surface; and
removing water to allow adhesion of the biocomposite to the region of the surface.

This invention relates to biosensors and methods and materials for use in manufacture of biosensors, particularly but not exclusively for automated or robotic manufacture of electrochemical sensors which incorporate biological materials.

Kumar, S. et al, Biosensors and Bioelectronics, 73, (2015), 114-122 discloses a reduced graphene oxide modified conducting paper for a cancer biosensor, in which conducting nanoparticles are deposited onto a porous paper substrate followed by physical absorption of anti-CEA onto the surface.

According to a first aspect of the present invention a method of manufacture of an electrochemical sensor precursor comprises the steps of:

preparing an adherent electrically conductive biocomposite comprising a mixture of a colloidal suspension in an aqueous liquid phase; the mixture including:
polycationic conductive polymer particles;
an anionic polyelectrolyte;
or a composite of a polycationic conducting polymer and an anionic polyelectrolyte
a biological affinity agent; and
an optional buffer;
applying the mixture to a region of an electroconductive surface; and
removing water to allow adhesion of the biocomposite to the region of the surface.

The biocomposite mixture may form a non-catalytic coating.

It has been unexpectedly found that biocompositse of this invention may adhere to suitable substrates, for example gold substrates. The biocomposites may not comprise covalent linkages between the biological agent and the polymer particles. The biocomposition mixture may be used as an ink.

The biosensors may be used in impedimetric assay systems using non-catalytic biological agents. These may be contrasted with enzymic or other catalytic reagents such as are used in voltammetric or amperometric systems.

The affinity agents may be selected from Affimers and antibodies. Affimers are engineered non-antibody binding proteins which may mimic molecular recognition characteristics of antibodies.

The systems of the present invention may be used to observe or measure binding of a target analyte to a biological affinity agent incorporated into the deposited biocomposition.

The present invention has the advantage of providing an all-in-one biocomposite which may be applied and adhere to an electrode substrate in a single step. This greatly simplifies manufacture of a biosensor in comparison to all previously disclosed systems.

A single liquid biocomposite may be provided, which may contain all of the required ingredients. This may facilitate pre-association of the biological and non-biological components to provide a stable, active composition that may be transported, stored and deposited as required to provide a stable bioactive film capable of specific recognition to give a signal reporting the presence of one or more target analytes.

The biocomposites may be transparent, facilitating use in photochromic devices or opaque. Both transparent and opaque biocomposites are effective in electrochemical devices, such as biosensors.

Examples of Target Analytes
Vascular biomarkers: LOX1
Cancer biomarkers: PSA, CA125, Bladder cancer FGFR3 and Colon Cancer CEA
Stroke biomarkers: S-100 protein, Neuron Specific Enolase.
Multiple Sclorosis biomarker: Myelin Basic Protein.
Cardiac biomarkers: Myoglobin, Troponin I, Haemoglobin, Atrial natriuretic peptide (ANP)
Inflammation/Infection Biomarker C-Reactive Protein (CRP)
Antibiotics and Drugs: Fluoroquinolines, sulphonamides and digoxin.
Herbicides Atrazine
Bacteria: Staphylococcus aureus, MRSA
   Clostridium difficile
   Group A Streptococcus (Streptococcus pyogenes)
   Serogroup B Meningococcus (colominic acid)
   E coli
   Neisseria gonorrhoea
   Gram −ve and Gram +ve Bacterial panels
Bacterial biomarker PBP2a
Other Proteins: human Chorionic Gonadotropin (hCG)
   human serum albumin (HSA)
Virus's Adenovirus 5

Examples of the required components are set out below, but not in any limitative sense.

1) Polycationic Conductive Polymer Particles

The polycationic conducting polymer may comprise particles having a dimension of about 5 to 750 nm, optionally about 10 to about 500 nm, optionally about 30 to about 400 nm, optionally about 70 to about 400 nm. The particles may comprise nanoparticles, nanotubes or mixtures thereof.

The polycationic conductive polymer may be selected from the group consisting of: polythiophenes and related polymers e.g. poly(3-methylthiophene), Poly(3-butylthiophene-2,5-diyl), poly(3-hexylthiophene) and the different regioregular polythiophenes e.g. regioregular poly(3-butylthiophene-2,5-diyl) and poly(3-hexylthiophene). Poly(3,4-ethylenedioxythiophene) (PEDOT) and related polymers e.g. poly (3,4-propylenedioxythiophene (PProDOT), and mixtures thereof. Alternative conductive polymers include polypyrrole and substituents thereof for example, poly (pyrrole-3-carboxilic acid), poly(l-cyanoethyl)pyrrole and poly(n-methyl)pyrrole), poly(polycarbazole and substituents thereof.

Alternative conductive polymers may be selected from the group consisting of polyaniline (PANI), and substituted polyanilines for example poly(2-aminobenzylamine), poly(2-aminobenzoic acid) and poly(2-methyl-aniline) and mixtures thereof.

Advantageous polycationic conductive polymers are selected from sulphur-containing polyheterocyclic compounds. Sulphur-containing cyclic coating compounds may form strong bonds to gold, platinum or other noble metal electrodes, with polythiophenes being some of the strongest.

2) Anionic Polyelectrolyte

The anionic polyelectrolyte may be selected from the group consisting of: polystyrene sulphonic acid (PSS), alginic acid, poly(acrylic acid), poly(vinyl sulphonic acid), dextran sulphate, heparin, and multi-anionic dyestuffs including direct red 80, direct blue 15, direct blue 14, direct blue 1, direct blue 6, acid blue 29, acid black 1, acid red 27, acid red 113 and mixtures thereof.

Exemplary anionic polyelectrolytes have 20 or more acid groups and may have a molecular weight greater than 20,000, for example 40,000 or greater.

3) Pre-Existing Composites of Polycationic Conducting Polymers and Anionic Polyelectrolytes

Pre-existing composites of Poly(3,4-ethylenedioxythiophene) PEDOT and Poly(styrenesulphonic acid) PSS are commercially available, for example from Bayer AG under the trade mark Baytron. A combination of PSS which is water soluble, with the insoluble PEDOT may give a material which acts as a solution but which comprises a dispersion of nanoparticles having various Redox states dependent on pH and other conditions. The measurement of particle size in 50 mM MOPS buffer at pH 7.05 provided particles of two sizes at about 38 nm and about 825 nm, although the larger particles could be aggregates. Dimensions of about 70 nm to about 400 nm have been obtained. Commercial PEDOT/PSS pre-existing composites for example as available from Sigma Aldrich, comprise 3 to 4 wt % in water at pH 5. Adding 50 mM, pH 7.5 MOPS buffer may effectively adjust the pH of the aqueous composite to 7. This may be a key step to incorporate active biological molecules.

Alternative pre-existing composites of polycationic conducting polymers and anionic polyelectrolytes include: polyaniline (PANI) nanoparticles and PSS, PANI/direct blue 15, PANI/direct red 80, PANI/acid red 27, Poly(2-methyl aniline) nanoparticles and PSS, Poly(2-methyl aniline)/direct blue 15, Poly(2-methyl aniline)/direct red 80, Poly(2-methyl aniline)/acid red 27, Polypyrrole nanoparticles and PSS, Polypyrrole/direct blue 15, Polypyrrole/direct red 80 and Polypyrrole/acid red 27.

Pre-existing composites of polythiophenes, e.g. PEDOT and PSS have an advantage of being very stable in electrochemical applications and may be used for more than 100 cycles without degradation.

4) Biological Affinity Agent

The biological affinity agent may be an antibody (polyclonal or monoclonal), antibody fragments such as F(ab) fragments, F(ab)₂ fragments or nanobodies (cloned Fv fragments), a binding protein such as cellulose binding protein, a nucleic acid such as DNE, RNA or mixtures thereof. In advantageous embodiments the affinity agent is an antibody biomimic such as an Affimer, Darpin or other conserved scaffold protein.

5) Optional Buffer

Buffers control the pH of aqueous solutions and form environments where biological affinity agents are most stable. The addition of a buffer into the biocomposite mixture is optional. Buffers include mixtures of phosphate salts e.g. disodium hydrogen phosphate and sodium dihydrogen phosphate, citric acid and sodium citrate, sodium tetraborate and boric acid. Advantageous buffers include those designated as ‘Good’ buffers, which are which are zwitterionic compounds, having a pKa value between 6 and 8. ‘Good’ buffers have low toxicity, are highly water-soluble, stable against pH changes, have hydrolytic and enzymatic stability and have less effects on biochemical reactions. They were first prepared by N. E. Good et al in 1966. Examples of ‘Good’ buffers include 3-(N-Morpholino)propanesulfonic acid or MOPS, 1,4-Piperazinediethanesulfonic acid or PIPES, 4-(2-Hydroxyethyl)piperazine-1-ethanesulfonic acid or HEPES and N-[Tris(hydroxymethyl)methyl]-3-aminopropanesulfonic acid or TAPS.

Addition of buffers into the biocomposite mixture may improve the adhesion of the biocomposite coating formed.

The addition of a biological affinity agent, for example an antibody protein to a PSS solution forms a complex due to electrostatic interaction between the protein and the PSS molecules to give a protein-polyelectrolyte complex. Addition of an antibody protein to a PEDOT:PSS nanoparticle suspension in an aqueous solution forms a complex mixture of the protein and the anionic (PSS) and cationic polymer (PEDOT) nanoparticles. It has been found that addition of such a protein/PEDOT/PSS nanoparticle biocomposite to an electroconductive surface, for example a screen printed gold electrode, has the unexpected result that the biocomposite adheres strongly to the surface and is not removed by washing with distilled water, buffer solutions or buffered non-ionic detergents such Tween 20 at a concentration of 0.1 v/v %. Furthermore addition of multiple layers of the protein/PEDOT/PSS nanoparticle biocomposite to a surface is possible to allow building up of a strongly adherent coating.

The biocomposition may be a nanocomposite material consisting of an aggregate or covalent complex of any of the above with a polymer molecule or nanoparticle (e.g. a gold nanoparticle-Affimer complex or gold nanoparticle-antibody complex or a dendrimer nanocomposite).

The method may further comprise the steps of application of one or more aliquots of the aqueous biocomposite to a selected region of the surface of an electrode, allowing the biocomposite to dry, forming a coating on the electrode. The step of adding the aqueous composite may be repeated as necessary to build up a layer on the surface.

The biocomposite may be applied to the substrate by drop casting, printing, spraying, using an air jet or other deposition method.

The electroconductive surface may be a region of an electroconductive electrode.

According to a second aspect of the present invention an electrochemical sensor precursor comprises an electroconductive surface comprising a plurality of electrodes, one electrode including a layer of an electrically conductive composite comprising polycationic conductive polymer particles, an anionic polyelectrolyte and a biological affinity agent.

The biological affinity agent is a non-catalytic agent as described above.

The electroconductive surface which may comprise an electrode may be composed of screen printed or ink jet printed carbon or noble metal, for example, gold or platinum.

Photolithographically or sputtered gold or platinum electrodes may be employed. However the use of photolithographically or sputtered gold electrodes may not be preferred on account of their high cost.

Use of a biosensor in accordance with the present invention confers numerous advantages. The biosensor has been found to function well in both Faradaic and non-Faradaic buffers, for example ferri/ferrocyanide buffer or in non-conductive buffers such as phosphate buffered saline (PBS). This unexpectedly provides the advantage that it allows an analyte solution to be added directly without modifying using a buffer or other additive solution. Also electropolymerization of the polymer coating onto the conductive surface is avoided, facilitating large scale robotic manufacture of arrays of the biosensors.

The biosensors may be employed in apparatus using AC impedance interrogation.

Alternatively or in addition or other methods to interrogate the non-catalytic coatings formed may include cyclic voltammetry, differential pulse voltammetry, chronopotentiometry and open circuit potentiometry.

The invention is further described by means of examples but not in any limitative sense with reference to the accompanying drawings of which:

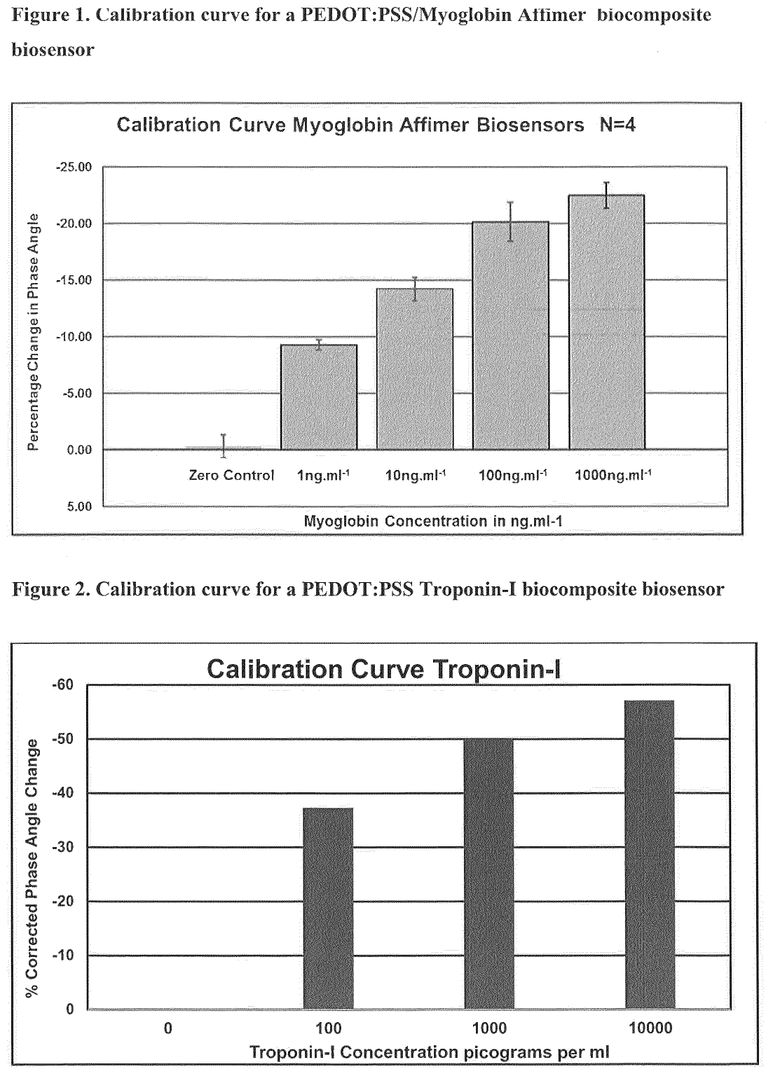

FIG. 1 shows a calibration curve for a PEDOT:PSS Myoglobin Affimer biocomposite biosensor;

FIG. 2 shows a calibration curve for a PEDOT:PSS Troponin-I biocomposite biosensor;

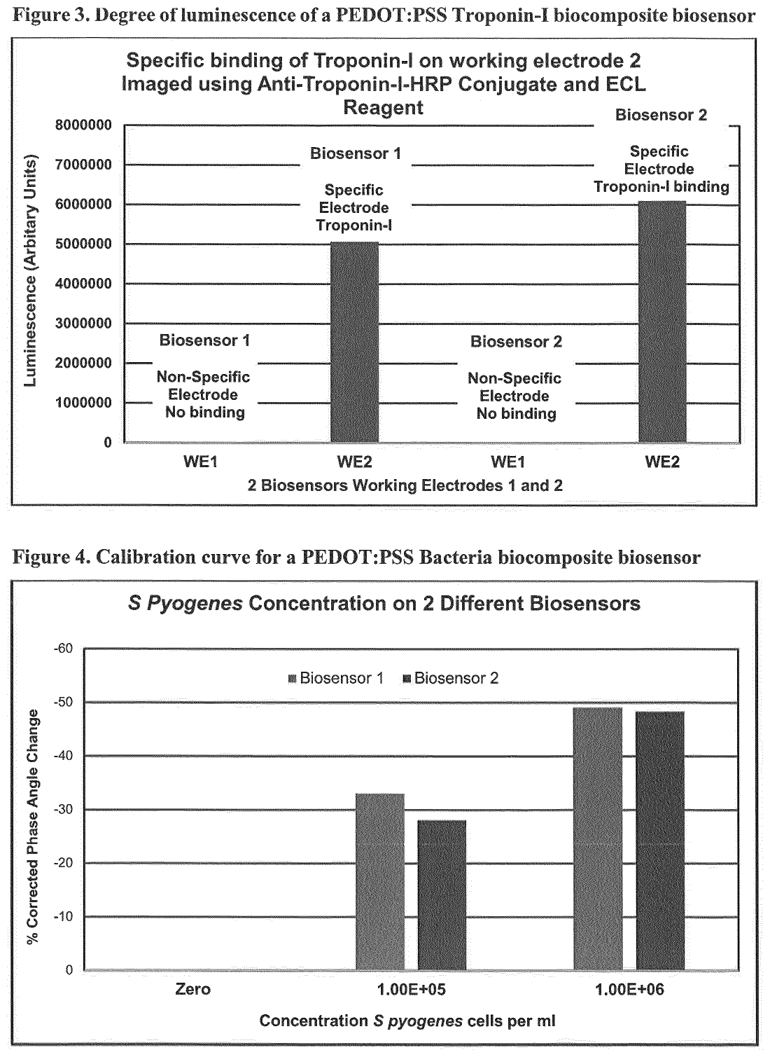

FIG. 3 shows the degree of luminescence of a PEDOT:PSS biocomposite biosensor;

FIG. 4 shows a calibration curve for a PEDOT:PSS Bacteria biocomposite biosensor;

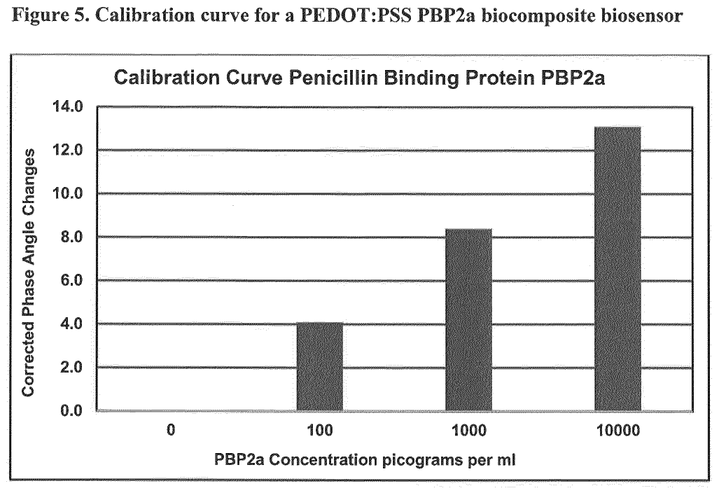

FIG. 5 shows a calibration curve for a PEDOT:PSS PBP2a biocomposite biosensor.

### EXAMPLE 1—PREPARATION OF PEDOT:PSS AFFIMER BIOCOMPOSITE AND CONSEQUENT Biosensor for Myoglobin Measurement

An Affimer biocomposite was prepared by the following method:

A 1 in 10 dilution of PEDOT:PSS nanoparticles (Sigma Aldrich catalogue number 655201, high conductivity grade 3-4% w/v dispersion in water) into deionised water was first made.

Then 10 μl of Anti-Human Myglobin Affimer (1 mg per ml in Affimer elution buffer pH 8.0 containing phosphate buffer, sodium chloride, imidazole and glycerol) was added to 40 μl of the 1/10 diluted PEDOT:PSS suspension, to give an Affimer/PEDOT:PSS biocomposite containing 200 μg per ml Affimer, designated as—specific Affimer complex or SAC.

A second Affimer biocomposite was prepared using Anti-ANP Affimer (1 mg per ml in 100 mM PBS buffer pH 7.1) adding to 40 μl of the 1/10 diluted PEDOT:PSS suspension, to give an Affimer/PEDOT:PSS biocomposite containing 200 μg per ml Affimer, designated as—non-specific Affimer complex or NSAC.

1.0 μl of SAC was deposited on the upper surface of working electrode 2 (WE2) of a dual gold electrode (DropSens X2220AT) and 1.0 μl or NSAC was deposited on the upper surface of working electrode 1 (WE1) of a dual gold electrode, then the deposited biocomposites were incubated at room temperature for 10 minutes and then dried on a hot plate set at 40° C. for a further 10 minutes. This procedure gave a thin adherent layer of Affimer/PEDOT:PSS biocomposite adhering strongly to the surface of the gold electrodes.

Incubation of solutions of Human Myoglobin onto both electrode surfaces for 10 minutes resulted in measurable changes to the AC impedance when they were scanned between 2500 Hz down to 0.25 Hz in ferri/ferrocyanide buffer (Faradaic buffer), indicating affinity binding took place. Any non-specific binding that occurred was corrected for by the NSAC on WE1 by subtracting the non-specific signal obtained from the signal from WE2. The differential signal (WE2-WE1) was a measure of the concentration of Human Myoglobin. A concentration dependant calibration curve is depicted in FIG. 1, where the results are normalized to a differential percentage impedance change. Four Affimer biocomposite biosensors gave good reproducibility as indicated by the error bars on the graph in FIG. 1.

### EXAMPLE 2—PREPARATION OF PEDOT:PSS BIOCOMPOSITE AND CONSEQUENT BIOSENSOR FOR TROPONIN-I MEASUREMENT

A biocomposite was prepared by the following method:

A 1 in 10 dilution of PEDOT:PSS nanoparticles (Sigma Aldrich catalogue number 655201, high conductivity grade 3-4% w/v dispersion in water) into 50 mM MOPS buffer pH 7.05 was first made.

Then 10 μl of polyclonal anti-troponin-I (1 mg per ml in 50 mM MOPS buffer pH 7.05) was added to 40 μl of the 1/10 diluted PEDOT:PSS suspension, to give an antibody/PEDOT:PSS biocomposite containing 200 μg per ml antibody, designated as—specific antibody complex or SAC.

A second antibody biocomposite was prepared using polyclonal anti-digoxin (2 mg per ml in 100 mM PBS buffer pH 7.1) adding to 90 μl of the 1/10 diluted PEDOT:PSS suspension, to give an antibody/PEDOT:PSS biocomposite containing 200 μg per ml antibody, designated as—non-specific antibody complex or NSAC.

0.4 μl of SAC was deposited on the upper surface of working electrode 2 (WE2) of a dual gold electrode (DropSens X2220AT) and 0.4 μl or NSAC was deposited on the upper surface of working electrode 1 (WE1) of a dual gold electrode, then the deposited suspensions were dried on a hot plate set at 37° C. This procedure was repeated 2 further times to give a thin layer of antibody/PEDOT:PSS biocomposite adhering strongly to the surface of the gold electrodes.

Incubation of solutions of Troponin-I onto both electrode surfaces for 5 minutes resulted in measurable changes to the AC impedance when they were scanned between 1000 Hz down to 1 Hz in PBS (non-Faradaic buffer), indicating affinity binding took place. Any non-specific binding that occurred was corrected for by the NSAC on WE1 by subtracting the non-specific signal obtained from the signal from WE2. The differential signal (WE2-WE1) was a measure of the concentration of Troponin-I. A concentration dependant calibration curve is depicted in FIG. 2.

### EXAMPLE 3—DEMONSTRATION OF ADHERENCE OF PROTEIN TO THE ELECTRODE SURFACE

Strong adherence of protein:PEDOT:PSS nanoparticle biocomposites to electrode surfaces and specific binding capabilities was demonstrated using an optical imaging technique. The electrode surfaces were prepared using a polyclonal Anti-Troponin-I added to PEDOT:PSS nanoparticles in MOPS buffer (45 mM, pH 7.05) to give an active biocomposite. Three aliquots of 0.2 μl were drop cast onto the electrode surfaces and dried at 37° C. for 2-3 minutes between aliquots. The control electrode was prepared using polyclonal Anti-Digoxin in exactly the same manner. The biosensors thus prepared were incubated with analyte (Troponin-I) and then washed with buffered 0.1% v/v Tween 20 to remove any non-specific bound analyte. Then an Anti-Troponin-HRP conjugate was used to label the bound analyte, washed again with buffered 0.1% v/v Tween 20 to remove any non-specific bound conjugate. Pierce ECL reagent was then added and the electrodes imaged in a GeneSys imager. The surfaces luminesced due to the bound HRP label. A higher degree of luminescence indicated bound analyte, FIG. 3.

### EXAMPLE 4—PREPARATION OF PEDOT:PSS BIOCOMPOSITE AND CONSEQUENT BIOSENSOR FOR STREPTOCOCCUS PYOGENES MEASUREMENT

A biocomposite was prepared by the following method:

A 1 in 10 dilution of PEDOT:PSS nanoparticles (Sigma Aldrich catalogue number 655201, high conductivity grade 3-4% w/v dispersion in water) into 50 mM MOPS buffer pH 7.05 was first made.

Then 10 μl of polyclonal anti-Streptococcus pyogenes (1 mg per ml in 50 mM MOPS buffer pH 7.05) was added to 40 μl of the 1/10 diluted PEDOT:PSS suspension, to give an antibody/PEDOT:PSS biocomposite containing 200 μg per ml antibody, designated as—specific antibody complex or SAC.

A second antibody biocomposite was prepared using polyclonal anti-digoxin (2 mg per ml in 100 mM PBS buffer pH 7.1) adding to 90 μl of the 1/10 diluted PEDOT:PSS suspension, to give an antibody/PEDOT:PSS biocomposite containing 200 μg per ml antibody, designated as—non-specific antibody complex or NSAC.

0.2 μl of 1 in 10 dilution of PEDOT:PSS nanoparticles was deposited on the upper surface of both working electrodes (WE1 and WE2) of a dual gold electrode (DropSens X2220AT) and dried on a hot plate set at 37° C. When dry 1 μl of NSAC was deposited on the upper surface of working electrode 1 (WE1) and 1 μl of SAC was deposited on the upper surface of working electrode 2 (WE2) of a dual gold electrode, then the deposited suspensions were dried on a hot plate set at 37° C. This 2 stage procedure gave a thin layer of antibody/PEDOT:PSS biocomposite adhering strongly to the surface of the gold electrodes.

Incubation of suspensions of Streptococcus pyogenes onto both electrode surfaces for 15 minutes resulted in measurable changes to the AC impedance when they were scanned between 1000 Hz down to 1 Hz in ferri/ferrocyanide buffer (Faradaic buffer), indicating affinity binding took place. Any non-specific binding that occurred was corrected for by the NSAC on WE1 by subtracting the non-specific signal obtained from the signal from WE2. The differential signal (WE2-WE1) was a measure of the concentration of Streptococcus pyogenes. A concentration dependant calibration curve is depicted in FIG. 4.

### EXAMPLE 5—PREPARATION OF PEDOT:PSS BIOCOMPOSITE AND CONSEQUENT BIOSENSOR FOR PENICILLIN BINDING PROTEIN 2A (PBP2A) MEASUREMENT. PBP2A IS A BIOMARKER FOR MRSA

A biocomposite was prepared by the following method:

A 1 in 10 dilution of PEDOT:PSS nanoparticles (Sigma Aldrich catalogue number 655201, high conductivity grade 3-4% w/v dispersion in water) into 50 mM MOPS buffer pH 7.05 was first made.

Then 10 μl of polyclonal anti-PBP2aI (1 mg per ml in 50 mM MOPS buffer pH 7.05) was added to 40 μl of the 1/10 diluted PEDOT:PSS suspension, to give an antibody/PEDOT:PSS biocomposite containing 200 μg per ml antibody, designated as—specific antibody complex or SAC.

A second antibody biocomposite was prepared using polyclonal anti-digoxin (2 mg per ml in 100 mM PBS buffer pH 7.1) adding to 90 μl of the 1/10 diluted PEDOT:PSS suspension, to give an antibody/PEDOT:PSS biocomposite containing 200 μg per ml antibody, designated as—non-specific antibody complex or NSAC.

0.4 μl of SAC was deposited on the upper surface of working electrode 2 (WE2) of a dual gold electrode (DropSens X2220AT) and 0.4 μl or NSAC was deposited on the upper surface of working electrode 1 (WE1) of a dual gold electrode, then the deposited suspensions were dried on a hot plate set at 37° C. This procedure was repeated 2 further times to give a thin layer of antibody/PEDOT:PSS biocomposite adhering strongly to the surface of the gold electrodes.

Incubation of solutions of PBP2a onto both electrode surfaces for 5 minutes resulted in measurable changes to the AC impedance when they were scanned between 1000 Hz down to 1 Hz in ferri/ferrocyanide buffer (Faradaic buffer), indicating affinity binding took place. Any non-specific binding that occurred was corrected for by the NSAC on WE1 by subtracting the non-specific signal obtained from the signal from WE2. The differential signal (WE2-WE1) was a measure of the concentration of PBP2a. A concentration dependant calibration curve is depicted in FIG. 5.

1. A method of manufacture of an electrochemical sensor biocomposite precursor comprising the steps of:
preparing an adherent electrically conductive biocomposite comprising a mixture of a colloidal suspension in an aqueous liquid phase, the mixture including:
polycationic conductive polymer particles;
an anionic polyelectrolyte or pre-existing composites of polycationic conducting polymers and anionic polyelectrolytes;
a biological affinity agent; and
an optional buffer;
applying the mixture to a region of an electroconductive surface; and
removing water to allow adhesion of the biocomposite to the region of the surface.

2. The method of claim 1, wherein the biocomposite is non-catalytic.

3. The method of claim 1, wherein the agent is an Affimer.

4. The method of claim 1, wherein the agent is an antibody.

5. The method of claim 1, wherein the polymer comprises particles having a dimension of 5 to 750 nm.

6. The method of claim 3, wherein the particles have a dimension of 10 to 500 nm.

7. The method of claim 4, wherein the particles have a dimension of 30 to 400 nm.

8. The method of claim 5, wherein the particles have a dimension of 70 to 400 nm.

9. The method of claim 5, wherein the particles comprise nanoparticles, nanotubes or mixtures thereof.

10. The method of claim 1, wherein the conductive polymer is selected from the group consisting of: poly (3,4-ethylenedioxythiophene), poly (3,4-propylenedioxythiophene, polypyrrole, substituted polypyrroles, poly(pyrrole-3-carboxylic acid), poly(1-cyanoethyl)pyrrole, poly(n-methyl)pyrrole), polycarbazole and substituted polycarbazoles.

11. The method of claim 1, wherein the conductive polymer is selected from the group consisting of: polyaniline (PANI) and mixtures thereof, substituted polyanilines, poly(2-aminobenzylamine), poly(2-aminobenzoic acid) and poly(2-methyl-aniline).

12. The method of claim 1, wherein the anionic polyelectrolyte is selected from the group consisting of:
polystyrene sulphonic acid (PSS), alginic acid, mixtures thereof and multi-anionic dyestuffs including direct red 80, direct blue 15, direct blue 14, direct blue 1, direct blue 6, acid blue 29, acid black 1, acid red 27, acid red 113 and mixtures thereof.

13. The method of claim 1, wherein the biological affinity agent is selected from the group consisting of:
a polyclonal antibody or monoclonal antibody, antibody fragments, F(ab) fragments, F(ab)2 fragments, nanobodies, a binding protein, cellulose binding protein, a nucleic acid, an antibody biomimic, Affimers or Darpins.

14. The method of claim 1, comprising the steps of application of aliquots of the aqueous mixture to a selected region of the surface of the electrode.

15. The method of claim 14, wherein the mixture is applied by drop casting, robotically controlled liquid deposition, printing or spraying.

16. An electrochemical sensor precursor comprising an electroconductive surface comprising a plurality of electrodes, one electrode including a layer of electrically conductive composite comprising polycationic conductive polymer particles and an anionic polyelectrolyte.

17. The electrochemical sensor precursor of claim 16, wherein the electroconductive surface comprises a gold, platinum, carbon or metal containing electrode produced by offset lithography printing, flexography printing, digital printing, xerography printing, gravure printing, screen printing, ink jet printing, photolithography or vapour deposition.

18. The impedometric sensor of claim 16.
